(19) United States
(12) Patent Application Publication
(10) Pub. No.: US 2012/0259393 A1
(43) Pub. Date: Oct. 11, 2012

(54) Implantable Device for Optically Stimulating the Brain of a Person or an Animal

(76) Inventors: Alim Louis Benabid, Meylan (FR); Guillaume Charvet, Sassenage (FR)

(21) Appl. No.: 13/273,598

(22) Filed: Oct. 14, 2011

(30) Foreign Application Priority Data
Apr. 8, 2011 (FR) 1101101

(51) Int. Cl. A61N 5/06

(52) U.S. Cl. 607/92

(57) ABSTRACT

The invention relates to an implantable device for optically stimulating the brain of a person or an animal, the device comprising:
a light source emitting in the near infrared range; and
a light guide that is implantable in the brain of a person or an animal, the light guide being provided with a sheath of biocompatible material and having a proximal end for receiving the light emitted by the source, and a distal end for delivering said light to the inside of the brain.

### FIELD OF THE INVENTION

The present invention relates to the field of deep stimulation of the brain of a human or an animal.

### BACKGROUND OF THE INVENTION

Deep brain stimulation is a therapeutic technique involving implanting a device for stimulating specific portions of the brain.

It is thus possible to improve various troubles, e.g. associated with depression, Alzheimer's disease, or Parkinson's disease.

In particular, the invention relates to the field of deep brain stimulation by optical irradiation.

Deep optical irradiation of the brain has already been proposed, in particular for treating Parkinson's disease.

For this purpose, proposals have already been made to irradiate biomolecular structures of the brain optically, and in particular to irradiate target cells that possess photosensitive proteins, with this being done by means of a device that is suitable for implanting in the brain (US 2009/0118800).

Nevertheless, the technique proposed in that document is relatively complex insofar as it implements a grid of openings through which light is or is not delivered to the brain, depending on selected parameters. When treating Parkinson's disease, the grid is used to determine the location in the brain to which light is to be sent in order to improve the treatment of the patient (cf. paragraph [0041] of that document). This provides a better profile for the stimulus that is to be applied to the patient under consideration. Without the grid, it can be understood that the treatment would probably be much less effective since it would not be known exactly where optical radiation is being sent to treat the patient.

Furthermore, the proteins of the cells targeted by the optical irradiation need to be provided with carrier ion channels of a zone that can be activated by optical irradiation (photosensitive proteins).

Another technique makes it possible to omit a step seeking to make the proteins of the target cells photosensitive with that technique.

Thus, the article by Victoria E. Shaw et al., “Neuroprotection of mid-brain dopaminergic cells in MPTP-treated mice after near-infrared light treatment”, J. of Comparative Neurology, 518:25-40 (2010), proposes optically irradiating a mouse with a lamp that emits in the near-infrared range in order to treat Parkinson's disease.

Nevertheless, the lamp needs to emit at relatively high power in order to irradiate all zones of the brain substantially. Such irradiation can give rise to undesirable side effects, such as burns or the destruction of certain cells. That technique is therefore difficult to envisage using in the treatment of an animal or a human being.

### OBJECT AND SUMMARY OF THE INVENTION

The invention proposes a solution for mitigating at least one of the above-mentioned drawbacks.

In order to achieve this object, the invention provides an implantable device for optically stimulating the brain of a person or an animal, the device comprising:
a light source emitting in the near infrared range; and
a light guide that is implantable in the brain of a person or an animal, the light guide being provided with a sheath of biocompatible material and having a proximal end for receiving the light emitted by the source, and a distal end for delivering said light to the inside of the brain.

The device may also provide other technical characteristics, taken singly or in combination:
diffuser means are provided at the distal end of the light guide for diffusing the light delivered by the light guide
the diffuser means are made of a material in which the reduced diffusion coefficient is greater than 0.1 per centimeter (cm⁻¹), preferably lying in the range 1 cm⁻¹ to 50 cm⁻¹, and the absorption coefficient is less than 10 cm⁻¹, and preferably less than 1 cm⁻¹;
the diffuser means present a dimension of less than 5 millimeters (mm) in a direction perpendicular to the longitudinal axis of the light guide, e.g. a dimension lying in the range 1 mm to 5 mm;
the light guide is a flexible light guide, such as an optical fiber;
the light source is arranged in such a manner as to emit light at a wavelength lying in the range 650 nanometers (nm) to 950 nm, e.g. 670 nm:
a confinement housing of biocompatible material is provided that is suitable for implanting in or on the skull or under the scalp of the person or animal and that includes at least coupler means for coupling the light emitted by the source with the proximal end of the light guide;
the light source is housed in the confinement housing;
the confinement housing also includes an internal control unit for controlling at least the intensity of the light emitted by the light source;
an external control unit is provided for controlling the internal control unit, said external control unit being designed to be applied in register with the confinement housing;
another confinement housing is provided that includes the light source, said other confinement housing being designed to be applied in register with the implantable confinement housing in such a manner that the coupler means can receive the light emitted by the light source;
the other confinement housing includes a control unit for controlling at least the intensity of the light emitted by the light source; and
releasable connection means are provided between said housing and the proximal end of the light guide.

### BRIEF DESCRIPTION OF THE DRAWINGS

Other characteristics, objects, and advantages of the invention are set out in the following detailed description made with reference to the following figures:

FIG. 1 shows a first embodiment of the invention; and

FIG. 2 shows a second embodiment of the invention.

### MORE DETAILED DESCRIPTION

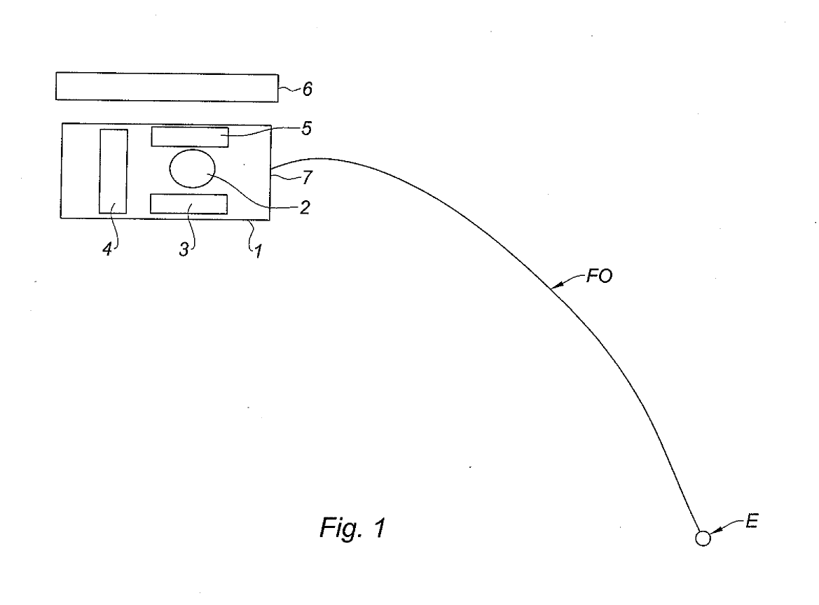

A first embodiment is shown diagrammatically in FIG. 1.

The optical irradiation device comprises a light source 2 emitting in the near infrared range. Typically, the wavelength emitted by the light source 2 lies in the range 650 nm to 950 nm, e.g. 670 nm.

The device also has a light guide FO comprising a proximal end and a distal end. The light guide FO is for implanting in the brain of a person or an animal in such a manner that its proximal end is suitable for receiving the light emitted by the light source 2, and the distal end of the guide is suitable for delivering the light inside the brain.

The light guide is provided with a sheath of biocompatible material.

The light guide may also present a diameter lying in the range 100 micrometers (μm) and a few millimeters, e.g. 5 mm.

Advantageously, the light guide FO is a flexible light guide such as an optical fiber. Under such circumstances, it may be made using a flexible polymer, preferably having a radius of curvature of at least 5 cm. A light guide that is flexible is easier to implant in certain zones of the brain.

Advantageously, the device also comprises means E for diffusing the optical radiation received by the light guide (FO) and located at the distal end of the light guide. This enables the radiation coming from the optical fiber to be diffused before it reaches brain tissues/cells. It is thus possible to use a light source that generates light at a relatively high density of power per unit area without running the risk of damaging brain tissues/cells.

The material forming the diffuser means E may be made of a material presenting the following properties: a reduced diffusion coefficient (μ′ₛ) that is greater than 0.1 cm⁻¹, preferably lying in the range 1 cm⁻¹ to 50 cm⁻¹, and an absorption coefficient (μₐ) is less than 10 cm⁻¹, preferably less than 1 cm⁻¹. Such a material may be a polymer including diffusing particles.

Advantageously, the diffuser means E are flexible and rounded. This serves to limit lesions in the tissue coming into contact with said means, and more generally in the tissue located at the distal end of the light guide FO. It is thus possible to envisage a material such as silicone for the diffuser means E.

Generally, the diffuser means are in the form of a sphere, e.g. having a diameter of less than 5 mm. In particular, it is possible to select a diameter lying in the range 1 mm to 5 mm. More generally, in a direction perpendicular to the longitudinal axis of the light guide FO, the diffuser means E should present a dimension of less than 5 mm, regardless of its shape.

The device also comprises a confinement housing 1 made of biocompatible material and suitable for implanting on or in the skull or under the scalp of the person or the animal. Preferably, the confinement housing 1 is for installing under the scalp.

The confinement housing 1 includes the light source 2, coupling means 4 such as a reflector for coupling the light emitted to the proximal end of the light guide 1, an internal control unit 5 suitable for controlling at least the intensity of the light emitted by the light source 2, and a battery 3 for powering the light source 2 and the internal control unit 5.

The internal control unit 5 may also control the emission of the radiation in the form of pulses, at a desired frequency. Pulsed emission serves to control the heating of the brain tissue that is to be irradiated.

Optionally, the device may include an external control unit 6 for controlling the internal control unit 5, the unit 6 being designed to be applied in register with the confinement housing 1. Under such circumstances, certain functions (or indeed all of the functions) performed by the internal control unit 5 may be transferred to the external control unit 6.

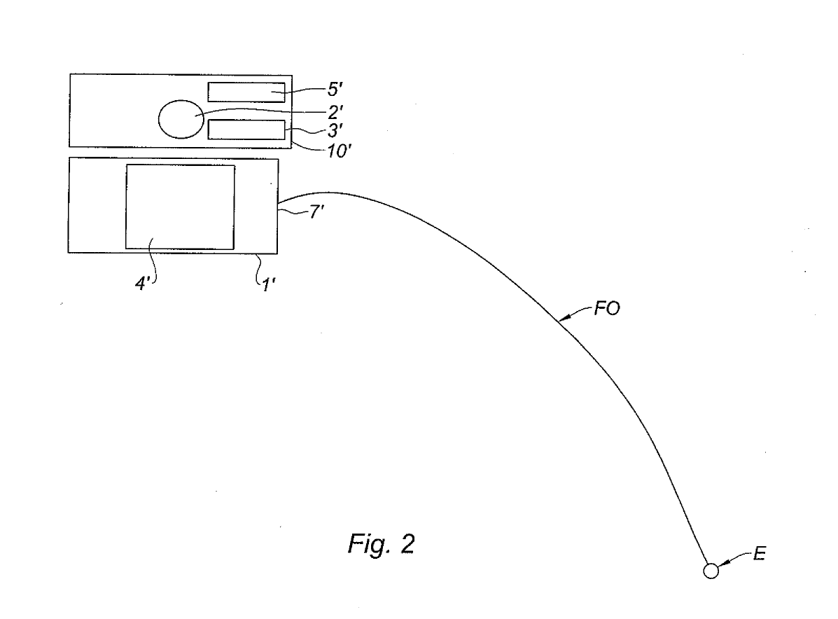

A second embodiment is shown diagrammatically in FIG. 2.

In this embodiment, the light source 2′, the control unit 5′ for controlling at least the intensity of the light emitted by the source 2′, and the battery 3′ are housed in another confinement housing 10′.

The implantable confinement housing 1′ thus contains no more than the coupling means 4′, e.g. formed by a reflector.

The light source 2′, the control unit 5′, and the coupling means 4′ may advantageously be identical respectively with the light source 2, the control unit 5, and the coupling means 4 described for the first embodiment.

Furthermore, all of the other means described for the first embodiment are transposable to the second embodiment. This applies in particular to the diffuser means that are to be installed at the distal end of the light guide.

The confinement housing 10′ is not designed to be implanted on the animal or the person under consideration. In operation, the confinement housing 10′ is thus applied in register with the implantable confinement housing 1′, in such a manner that the coupling 4′ can receive the optical radiation emitted by the light source 2′.

The advantage of this embodiment lies in the fact that only passive means (reflector, light guide, . . . ) then need to be implanted in the brain of the animal or the person being treated.

Thus, it is possible to implant all of the passive means in the animal or the person to be treated in a definitive manner and to bring the confinement housing 10′ into position only when a treatment session is needed.

In a variant, it is also possible to provide connection means (not shown) between the two confinement means 1′, 10′ for the purpose of reversibly connecting the confinement means 1′ and 10′ together. No surgical intervention is then needed. For example, if it is desired to change the battery 3′, it then suffices to disconnect the other confinement housing 10′ from the confinement housing 1′, to open said other confinement housing 10′, to change the battery 3′, and finally to reconnect the two confinement means 1′, 10′ together.

In a version covering both embodiments of the invention, an implantable obstacle irradiation device is thus proposed that comprises:
a flexible longitudinal light guide for inserting in the brain, in such a manner that its distal end is suitable for delivering optical radiation to the inside of the brain;
an implantable confinement housing; and
connection means for providing a reversible connection between the light guide and the confinement housing.

This device may have the characteristics described above.

There follows a description of an optical irradiation method that can be envisaged with a device in accordance with the invention.

A method of optically stimulating the brain of a person or an animal, wherein:

a) light is emitted in the near infrared range;

b) said light is delivered to a proximal end of a light guide that is implanted in the brain of the person or the animal; and

c) said light is guided along said light guide to a distal end thereof, in such a manner that the light irradiates the inside of the brain from said distal end.

The method may also include one or more of the following steps:
the light emitted in step a) is emitted in a wavelength range of 650 nm to 950 nm, e.g. 670 nm;
with the device including means (E) for diffusing the light at the distal end of the light guide (FO), the inside of the brain is irradiated via said means (E);
step b) of delivering the light to the light guide is performed via coupling means (4) for coupling the emitted light to the light guide (FO);
the method includes a step in which the intensity of the emitted light is controlled, and optionally also the frequency at which said light is emitted when pulsed light is to be delivered to the light guide;
the distal end of the light guide (FO) is implantable close to the compact black substance (SNc or “Substancia Nigra pars compacta”) of the brain;
the distal end of the light guide (FO) is implanted in the third ventricle of the brain, which ventricle is situated close to the compact black substance; and
the distal end of the light guide (FO) is implanted close to or in contact with the floor of the third ventricle of the brain.

It should be observed that the optical radiation emitted in the near infrared reaching the compact black substance limits degradation of dopaminergic cells. It is cell degradation that is implicated in Parkinson's disease.

Thus, the optical irradiation method of the invention comprises a step of therapeutic treatment for Parkinson's disease.

Furthermore, implanting the light guide in the third ventricle of the brain serves to approach the compact black substance while minimizing any risk of lesions caused to the brain matter surrounding said substance.

The fact of implanting the distal end of the light guide close to or even in contact with the floor of the third ventricle is particularly advantageous since it limits a little more any risk of lesions of brain matter. Under such circumstances, the distance between the distal end of the light guide and the compact black substance is about 2 centimeters (cm) in a human being, so the ventricles of the brain are less likely to be irradiated.

In order to implant the distal end of the light guide in the third ventricle, prior to implementing step a), the light guide is introduced into the third ventricle via the Monro hole.

This introduction may be performed using a cannula serving as a guide for the light guide, the cannula being withdrawn after the light guide has been implanted. The cannula (not shown) may be in the form of a hollow cylinder, with a lateral opening at one of its ends in order to enable the light guide to exit. A detailed description of the procedure for implanting a probe for deep brain stimulation is to be found in document “Medtronic—DBS™ lead kit for deep brain stimulation 3387 3389—Implant manual”, published by the supplier Medtronic Inc., downloadable from the Internet site: http://www.medtronic.com/physician/activa/downloadablefiles/197928_b_{—}006.pdf. The procedure that may be used in the context of the invention may essentially reproduce the procedure described in that document.

In human beings, the Monro hole presents a diameter that generally lies in the range 3 mm to 6 mm. When the light guide FO is placed in the third ventricle of a human being, passing via the Monro hole, it is therefore necessary to use a light guide of diameter that does not exceed 6 mm, or even 3 mm, depending on circumstances.

More generally, the person skilled in the art will adapt the diameter of the light guide to the diameter of the Monro hole.

In order to reach compact black surfaces in both hemispheres of the brain, two light guides may thus be implanted as described above in the proximity of each of the two compact black surfaces.

Nevertheless, it is advantageous to implant only one light guide. The fact of applying radiation via an endoventricular route makes it possible with a single light guide to reach the compact black substance in both hemispheres of the brain.

Finally, it should be observed that mammals, birds, and reptiles are all animals possessing compact black substance, close to which the light guide can be implanted.

1. An implantable device for optically stimulating the brain of a person or an animal, the device comprising:
a light source emitting in the near infrared range; and
a light guide that is implantable in the brain of a person or an animal, the light guide being provided with a sheath of biocompatible material and having a proximal end for receiving the light emitted by the source, and a distal end for delivering said light to the inside of the brain.

2. A device according to claim 1, wherein diffuser means are provided at the distal end of the light guide for diffusing the light delivered by the light guide.

3. A device according to claim 2, wherein the diffuser means are made of a material in which:
the reduced diffusion coefficient is greater than 0.1 cm⁻¹, preferably lying in the range 1 cm⁻¹ to 50 cm⁻¹; and
the absorption coefficient is less than 10 cm⁻¹, and preferably less than 1 cm⁻¹.

4. A device according to claim 2, wherein the diffuser means present a dimension of less than 5 mm in a direction perpendicular to the longitudinal axis of the light guide, e.g. a dimension lying in the range 1 mm to 5 mm.

5. A device according to claim 1, wherein the light guide is a flexible light guide, such as an optical fiber.

6. A device according to claim 1, wherein the light source is arranged in such a manner as to emit light at a wavelength lying in the range 650 nm to 950 nm, e.g. 670 nm.

7. A device according to claim 1, wherein a confinement housing of biocompatible material is provided that is suitable for implanting in or on the skull or under the scalp of the person or animal and that includes at least coupler means for coupling the light emitted by the source with the proximal end of the light guide.

8. A device according to claim 7, wherein the light source is housed in the confinement housing.

9. A device according to claim 8, wherein the confinement housing also includes an internal control unit for controlling at least the intensity of the light emitted by the light source.

10. A device according to claim 9 wherein an external control unit is provided for controlling the internal control unit, said external control unit being designed to be applied in register with the confinement housing.

11. A device according to claim 7, wherein another confinement housing is provided that includes the light source, said other confinement housing being designed to be applied in register with the implantable confinement housing in such a manner that the coupler means can receive the light emitted by the light source.

12. A device according to claim 11, wherein the other confinement housing includes a control unit for controlling at least the intensity of the light emitted by the light source.

13. A device according to claim 7, wherein releasable connection means are provided between said housing and the proximal end of the light guide.

14. A method of optically stimulating the brain of a person or an animal, wherein:
a) light is emitted in the near infrared range;
b) said light is delivered to a proximal end of a light guide that is implanted in the brain of the person or the animal; and
c) said light is guided in said light guide to a distal end thereof, in such a manner that the light irradiates the inside of the brain from said distal end.

15. A method according to claim 14, wherein the light emitted during step a) is emitted in a wavelength range of 650 nm to 950 nm, e.g. 670 nm.

16. A method according to claim 14, wherein the inside of the brain is irradiated via diffuser means for diffusing light that are situated at the distal end of the light guide.

17. A method according to claim 14, wherein step b) of delivering light to the light guide is performed via coupling means for coupling the emitted light to the light guide.

18. A method according to claim 14, wherein a step is provided in which the intensity of the emitted light is controlled, and optionally also the frequency at which said light is emitted when pulsed light is to be delivered to the light guide.

19. A method according to claim 14, wherein the distal end of the light guide is implanted close to the compact black substance of the brain.

20. A method according to claim 14, wherein the distal end of the light guide is implanted in the third ventricle of the brain, which ventricle is situated close to the compact black substance.

21. A method according to claim 14, wherein the distal end of the light guide is implanted close to or in contact with the floor of the third ventricle of the brain.

22. A method according to claim 14, wherein there is provided a step of treating Parkinson's disease.

23. A method of treating Parkinson's disease by optically stimulating the brain of a person or an animal, wherein:
a) light is emitted in the near infrared range;
b) said light is delivered to a proximal end of a light guide that is implanted in the brain of the person or the animal; and
c) said light is guided in said light guide to a distal end thereof, in such a manner that the light irradiates the inside of the brain from said distal end.
